(19) United States
(12) Patent Application Publication
(10) Pub. No.: US 2004/0161795 A1
(43) Pub. Date: Aug. 19, 2004

(54) Systems and methods for analysis of protein post-translational modification

(75) Inventor: Jarrod A. Marto, Charlottesville, VA (US)

Correspondence Address: ROPES & GRAY LLP, ONE INTERNATIONAL PLACE, BOSTON, MA, 02110-2624, US

(73) Assignee: MDS Proteomics Inc., Toronto (CA)

(21) Appl. No.: 10/627,309

(22) Filed: Jul. 25, 2003

Related U.S. Application Data
(60) Provisional application No. 60/398,682, filed on Jul. 25, 2002.

(51) Int. Cl. G01N 33/53; C12Q 1/37

(52) U.S. Cl. 435/7.1; 435/23

(57) ABSTRACT

The invention relates to a method for the detection and identification of amino acid modifications, such as phosphorylation, using a combination of affinity capture and mass-spectroscopy.

### REFERENCE TO RELATED APPLICATIONS

This application claims the benefit of earlier filing date, under 35 U.S.C. 119(e), of U.S. Provisional Application 60/398,682, filed on Jul. 25, 2002, the entire content of which is incorporated herein by reference.

### FIELD OF THE INVENTION

The invention relates to a method for the detection and identification of amino acid modifications, such as phosphorylation, using a combination of affinity capture and mass-spectroscopy.

### BACKGROUND TO THE INVENTION

With the availability of a burgeoning sequence database, genomic applications demand faster and more efficient methods for the global screening of protein expression in cells. However, the complexity of the cellular proteome expands substantially if protein post-translational modifications are also taken into account.

Dynamic post-translational modification of proteins is important for maintaining and regulating protein structure and function. Among the several hundred different types of post-translational modifications characterized to date, protein phosphorylation plays a prominent role. Enzyme-catalyzed phosphorylation and dephosphorylation of proteins is a key regulatory event in the living cell. Complex biological processes such as cell cycle, cell growth, cell differentiation, and metabolism are orchestrated and tightly controlled by reversible phosphorylation events that modulate protein activity, stability, interaction and localization. Perturbations in phosphorylation states of proteins, e.g. by mutations that generate constitutively active or inactive protein kinases and phosphatases, play a prominent role in oncogenesis. Comprehensive analysis and identification of phosphoproteins combined with exact localization of phosphorylation sites in those proteins (“phosphoproteomics”) is a prerequisite for understanding complex biological systems and the molecular features leading to disease.

It is estimated that ⅓ of all proteins present in a mammalian cell are phosphorylated and that kinases, enzymes responsible for that phosphorylation, constitute about 1-3% of the expressed genome. Organisms use reversible phosphorylation of proteins to control many cellular processes including signal transduction, gene expression, the cell cycle, cytoskeletal regulation and apoptosis. A phosphate group can modify serine, threonine, tyrosine, histidine, arginine, lysine, cysteine, glutamic acid and aspartic acid residues. However, the phosphorylation of hydroxyl groups at serine (90%), threonine (10%), or tyrosine (0.05%) residues are the most prevalent, and are involved among other processes in metabolism, cell division, cell growth, and cell differentiation. Because of the central role of phosphorylation in the regulation of life, much effort has been focused on the development of methods for characterizing protein phosphorylation.

The identification of phosphorylation sites on a protein is complicated by the facts that proteins are often only partially phosphorylated and that they are often present only at very low levels. Therefore techniques for identifying phosphorylation sites should preferably work in the low picomole to sub-picomole range.

Traditional methods for analyzing O-phosphorylation sites involve incorporation of ³²P into cellular proteins via treatment with radiolabeled ATP. The radioactive proteins can be detected during subsequent fractionation procedures (e.g. two-dimensional gel electrophoresis or high-performance liquid chromatography [HPLC]). Proteins thus identified can be subjected to complete hydrolysis and the phosphoamino acid content determined. The site(s) of phosphorylation can be determined by proteolytic digestion of the radiolabeled protein, separation and detection of phosphorylated peptides (e.g. by two-dimensional peptide mapping), followed by peptide sequencing by Edman degradation. These techniques can be tedious, require significant quantities of the phosphorylated protein and involve the use of considerable amounts of radioactivity.

In recent years, mass spectrometry (MS) has become an increasingly viable alternative to more traditional methods of phosphorylation analysis. The most widely used method for selectively enriching phosphopeptides from mixtures is immobilized metal affinity chromatography (IMAC). In this technique, metal ions, usually Fe³⁺ or Ga³⁺, are bound to a chelating support. Phosphopeptides are selectively bound because of the affinity of the metal ions for the phosphate moiety. The phosphopeptides can be released using high pH or phosphate buffer, the latter usually requiring a further desalting step before MS analysis. Limitations of this approach include possible loss of phosphopeptides because of their inability to bind to the IMAC column, difficulty in the elution of some multiply phosphorylated peptides, and background from unphosphorylated peptides (typically acidic in nature) that have affinity for immobilized metal ions. Two types of chelating resin are commercially available, one using iminodiacetic acid and the other using nitrilotriacetic acid. Some groups have observed that iminodiacetic acid resin is less specific than nitrilotriacetic acid, whereas another study reported little difference between the two. Several studies have examined off-line MS analysis of IMAC-separated peptides.

Recently, two groups have described protocols to achieve this goal. Oda et al. (Nat Biotechnol. 2001 19:379-82) start with a protein mixture in which cysteine reactivity is removed by oxidation with performic acid. Base hydrolysis is used to induce elimination of phosphate from phosphoserine and phosphothreonine, followed by addition of ethanedithiol to the alkene. The resulting free sulfhydryls are coupled to biotin, allowing purification of phosphoproteins by avidin affinity chromatography. Following elution of phosphoproteins and proteolysis, enrichment of phosphopeptides is carried out by a second round of avidin purification. Disadvantages of this approach include the failure to detect phosphotyrosine containing peptides and generation of diastereoisomers in the derivatization step.

The approach suggested by the Zhou et al. (Nat Biotechnol 2001 19:375-378) circumvents these problems but involves a six step derivatization/purification protocol for tryptic peptides that requires more than 13 hrs to complete and affords only a 20% yield from picomoles of phosphopeptide starting material. The method begins with a proteolytic digest that has been reduced and alkylated to eliminate reactivity from cysteine residues. Following N-terminal and C-terminal protection, phosphoramidate adducts at phosphorylated residues are formed by carbodiimide condensation with cystamine. The free sulfhydryl groups produced from this step are covalently captured onto glass beads coupled to iodoacetic acid. Elution with trifluoroacetic acid then regenerates phosphopeptides for analysis by mass spectrometry.

### SUMMARY OF THE INVENTION

One aspect of the present invention provides a method for identifying modified amino acids within a protein by combining affinity purification and mass spectroscopy in a manner which is amenable to high throughput and automation. In general, the subject method makes use of affinity capture reagents for isolating, from a protein sample, those proteins which have been post-translationally modified with a moiety of interest. In order to improve the selectivity/efficiency of the affinity purification step, proteins of the protein samples to be analyzed may be additionally chemically modified at at least one of: the C-terminal carboxyl, the N-terminal amine, and at least one of the amino acid side chains of the proteins which may interfere with the selectively of the affinity purification step for the post-translational modification of interest. Proteins which are isolated based on post-translational modifications are than analyzed by mass spectroscopy in order to identify patterns of modification across a proteome, and/or to provide the identity of proteins in the sample which are modified or shows changes in modification status between two different samples.

Thus one aspect of the invention provides a method for identifying modified amino acids within a protein, comprising: (i) providing one or more samples and an affinity capture reagent for isolating, from said samples, those proteins post-translationally modified by a moiety of interest; (ii) processing said samples to chemically modify at least one of the C-terminal carboxyl, the N-terminal amine and amino acid side chains of polypeptides in said samples so as to increase the specificity of said affinity capture reagent for those proteins post-translationally modified by said moiety of interest; (iii) isolating said proteins post-translationally modified by said moiety of interest from said samples using said affinity capture reagent; (iv) eluting said proteins bound to said affinity capture reagent by manipulating the oxidation state of said affinity capture reagent; and, (v) determining the identity of said proteins eluted in (iv) by mass spectroscopy.

In one embodiment, said polypeptides in said samples are further cleaved into smaller peptide fragments before, after or during the step of processing said samples. For instance, the proteins can be fragmented by enzymatic hydrolysis to produce peptide fragments having carboxy-terminal lysine or arginine residues. In certain preferred embodiments, the proteins are fragmented by treatment with trypsin.

In certain embodiments, the proteins are mass-modified with isotopic labels before, after or during the chemical modification step.

In one embodiment, isolated proteins are further separated by reverse phase chromatography before analysis by mass spectroscopy.

In one embodiment, isolated proteins are identified from analysis using tandem mass spectroscopy techniques.

In one embodiment, the identity of the eluted proteins are determined by searching molecular weight databases for the molecular weight observed by mass spectroscopy for an isolated protein or peptide fragment thereof.

In one embodiment, the method further comprises obtaining amino acid sequence mass spectra for said proteins or peptide fragments thereof, and searching one or more sequence databases for the sequence(s) observed for said protein or peptide fragments thereof.

In one embodiment, the moiety of interest is a phosphate group.

In one embodiment, the affinity capture reagent is an immobilized metal affinity chromatography medium, and step (ii) includes chemically modifying the side chains of glutamic acid and aspartic acid residues to neutral derivatives.

In one embodiment, the side chains of glutamic acid and aspartic acid residues are modified by alkyl-esterification.

In one embodiment, the sample comprises a mixture of different proteins.

In one embodiment, the sample is derived from a biological fluid, or a cell or tissue lysate.

In one embodiment, the method is conducted in two or more different samples, and the polypeptides or fragments thereof of each sample are isotopically labeled in a manner which permits discrimination of mass spectroscopy data between different samples.

In another aspect of the invention, peptides bound to the affinity capture reagent are eluted by manipulation of the oxidation state of the affinity capture reagent, such that the bound peptides have a lower affinity for the resultant oxidation state and, therefore, elute off the column. After elution of the peptides of interest, the affinity column is regenerated using a suitable redox reagent to return it to its original oxidation state.

There are a variety of mass spectroscopy techniques which can be employed in the subject method. In certain preferred embodiments, the isolated proteins are identified from analysis using tandem mass spectroscopy techniques, such as LC/MS/MS. Where the proteins have been further fragmented with trypsin or other predictable enzymes, the molecular weight of a fragment as determined from the mass spectroscopy data can be used to identify possible matches in molecular weight databases indexed by predicted molecular weights of protein fragments which would result under similar conditions as the fragments generated in the subject method. However, the subject method can be carried out using mass spectroscopy techniques which produce amino acid sequence mass spectra for the isolated proteins or peptide fragments. The sequence data can be used to search one or more sequence databases.

In certain preferred embodiments, the method is used to identify phosphorylated proteins or changes in the phosphorylation pattern amongst a group of proteins. In such embodiments, the affinity capture reagent can be an immobilized metal affinity chromatography medium, and the step of processing the protein samples includes chemically modifying the side chains of glutamic acid and aspartic acid residues to neutral derivatives, such as by alkyl-esterification.

It is contemplated that all embodiments described above may be combined whenever appropriate.

The subject method is amenable to analysis of multiple different protein samples, particularly in a multiplex fashion. In such embodiments, the proteins or fragments thereof are isotopically labeled in a manner which permits discrimination of mass spectroscopy data between protein samples. That is, a mass spectra on the mixture of various protein samples can be deconvoluted to determine the sample origin of each signal observed in the spectra. In certain embodiments, this technique can be used to quantitate differences in phosphorylation (or other modification) levels between samples prepared under different conditions and admixed prior to MS analysis.

In certain embodiments, the subject method is used for analyzing a phosphoproteome. For example, the proteins in the sample can be chemically modify at glutamic acid and aspartic acid residues, such as by alkyl-esterification, to generate neutral side chains at those positions. The phosphorylated proteins in the same are then isolated by immobilized metal affinity chromatography, and analyzed by mass spectroscopy. In preferred embodiments, the proteins are cleaved, e.g., by trypsin digestion or the like, into smaller peptide fragments before, after or during the step of chemically modify the glutamic acid and aspartic acid residues. In one embodiment, the subject method is carried out on multiple different protein samples, and proteins which a differentially phosphorylated between two or more protein samples are identified. That data can, for instance, be used to generate or augment databases with the identity of proteins which are determined to be phosphorylated.

Thus this aspect of the invention provides a method for analyzing a phosphoproteome, comprising: (i) providing one or more protein sample(s); (ii) chemically modifying the side chains of glutamic acid and aspartic acid residues of polypeptides in said protein sample(s) to neutral derivatives; (ii) isolating phosphorylated proteins from said protein sample(s) by using immobilized metal affinity chromatography; (iii) eluting said phosphorylated proteins from said affinity capture reagent by manipulating the oxidation state of said reagent; and, (iv) determining the identity of said phosphorylated proteins eluted in (iii) by mass spectroscopy.

In one embodiment, the method further comprises cleaving said polypeptides into smaller peptide fragments, before, after or during the step of chemically modifying the glutamic acid and aspartic acid residues.

In one embodiment, the polypeptides are fragmented by enzymatic hydrolysis to produce peptide fragments having carboxy-terminal lysine or arginine residues.

In one embodiment, the polypeptides are fragmented by treatment with trypsin.

In one embodiment, the glutamic acid and aspartic acid residues are modified by alkyl-esterification.

In one embodiment, said one or more sample(s) comprise two or more different samples, the method further comprises identifying proteins which are differentially phosphorylated between said two or more different samples.

In one embodiment, the method further comprises generating or adding to a database the identity of proteins which are determined to be phosphorylated.

Another aspect of the invention provides a method for identifying a treatment that modulates a modification of amino acid in a target polypeptide. In general, this method is carried out by providing a protein sample which has been subjected to a treatment of interest, such as treatment with ectopic agents (drugs, growth factors, etc). The protein samples can also be derived from normal cells in different states of differentiation or tissue fate, or derived from normal and diseased cells. Following the affinity purification/MS method set forth above, the identity of proteins which are differentially modified in the treated protein sample relative to an untreated sample or control sample can determined. From this identification step, one can determine whether the treatment results in a pattern of changes in protein modification, relative to the untreated sample or control sample, which meet a pre-selected criteria. Thus, one can use this method to identify compounds likely to mimic the effect of a growth factor by scoring for similarities in phosphorylation patterns when comparing proteins from the compound-treated cells with proteins from the growth factor treated cells. The treatment of interest can include contacting the cell with such compounds as growth factors, cytokines, hormones, or small chemical molecules. In certain embodiments, the method is carried out with various members of a chemically diverse library.

Thus this aspect of the invention provides a method for identifying a treatment that modulates a modification of amino acid in a target polypeptide, comprising: (i) providing a sample which has been subjected to a treatment of interest; (ii) determining, using the method of claim 1, the identity of proteins which are differentially modified in said treated sample relative to an untreated sample or control sample; (iii) determining, whether said treatment results in a pattern of changes in protein modification which meets a preselected criterion, in said treated sample relative to said untreated sample or control sample.

In one embodiment, the treatment is effected by a compound.

In one embodiment, the compound is a growth factor, a cytokine, a hormone, or a small chemical molecule.

In one embodiment, the compound is from a chemical library.

In one embodiment, the sample is derived from a cell or tissue subjected to said treatment of interest.

Yet another aspect of the present invention provides a method of conducting a drug discovery business. Using the assay described above, one determines the identity of a compound that produces a pattern of changes in protein modification, relative to the untreated sample or control sample, which meet a preselected criteria. Therapeutic profiling of the compound identified by the assay, or further analogs thereof, can be carried out for determining efficacy and toxicity in animals. Compounds identified as having an acceptable therapeutic profile can then be formulated as part of a pharmaceutical preparation. In certain embodiments, the method can include the additional step of establishing a distribution system for distributing the pharmaceutical preparation for sale, and may optionally include establishing a sales group for marketing the pharmaceutical preparation. In other embodiments, rather than carry out the profiling and/or formulation steps, one can license, to a third party, the rights for further drug development of compounds that are discovered by the subject assay to alter the level of modification of the target polypeptide.

Thus this aspect of the invention provides a method of conducting a drug discovery business, comprising: (i) determining, by any one of the above suitable methods, the identity of a compound that produces a pattern of changes in protein modification which meet a preselected criterion, in said treated sample relative to said untreated sample or control sample; (ii) conducting therapeutic profiling of said compound identified in step (i), or further analogs thereof, for efficacy and toxicity in animals; and, (iii) formulating a pharmaceutical preparation including one or more compound(s) identified in step (ii) as having an acceptable therapeutic profile.

This aspect of the invention also provides a method of conducting a drug discovery business, comprising: (i) determining, by the method of claim 24, the identity of a compound that produces a pattern of changes in protein modification which meet a preselected criterion, in said treated sample relative to said untreated sample or control sample; (ii) licensing, to a third party, the rights for further drug development of compounds that alter the level of modification of the target polypeptide.

Yet another aspect of the present invention provides a method of conducting a drug discovery business in which, after determining the identity of a protein that is post-translationally modified under the conditions of interest, the identity of one or more enzymes which catalyze the post-translational modification of the identified protein under the conditions of interest is determined. Those enzyme(s) are then used as targets in drug screening assays for identifying compounds which inhibit or potentiate the enzymes and which, therefore, can modulate the post-translational modification of the identified protein under the conditions of interest.

Thus this aspect of the invention provides a method of conducting a drug discovery business, comprising: (i) by the method of claim 1, determining the identity of a protein that is post-translationally modified under conditions of interest; (ii) identify one or more enzymes which catalyze the post-translational modification of the identified protein under the conditions of interest; (iii) conduct drug screening assays to identify compounds which inhibit or potentiate the enzymes identified in step (ii) and which modulate the post-translational modification of the identified protein under the conditions of interest.

### REFERENCE TO THE DRAWINGS

FIG. 1 shows data acquired for a simple standard peptide (angiotensin II phosphate). The phospho-peptide in the figure (DRVpYIHPF) is represented by SEQ ID NO: 1.

FIG. 2 shows enrichment of phosphorylated peptides from a complex biological mixture. The data illustrates the MS and MS/MS spectra acquired for a phosphorylated peptide from a human lamin protein. The phospho-peptide in the figure (ASpSHSSQTQGGGSVTK) is represented by SEQ ID NO: 2.

FIG. 3 is a schematic drawing of an exemplary system for automating one embodiment of the subject method.

### DETAILED DESCRIPTION OF THE INVENTION

The current progression from genomics to proteomics is fueled by the realization that many properties of proteins (e.g., interactions, post-translational modifications) cannot be predicted from DNA sequence. The present invention provides a method useful to identify modified amino acid sites within peptide analytes. These modified amino acids are amino acids that incorporate conjugating groups including but not limited to those conjugating groups are that incorporated naturally by the cell, typically as post-translational modifications. Such conjugating groups include saccharide moieties, such as monosaccharides, disaccharides and polysaccharides. Such conjugating groups further include lipids and glycosaminoglycans. Other modified amino acids containing various types of conjugating groups can also be detected by the present method, including amino acids modified by iodination, bromination, nitration and sulfation, and particularly amino acids modified by phosphorylation. In certain preferred embodiments, the subject method is used to identify phosphate modified serine, threonine, tyrosine, histidine, arginine, lysine, cysteine, glutamic acid and aspartic acid residues, more preferably to identify phosphoserine, phosphothreonine and phosphotyrosine-containing peptides.

The subject invention provides apparatus and methods for automating the use of mass spectroscopy for identifying post-translationally modified polypeptides. In particular, the subject method provides for automation of a process including affinity chromatography capture of post-translationally modified proteins, and processing the modified proteins for analysis by mass spectroscopy. Unlike the prior art methods which require conversion of the modified amino acid residue to another chemical entity which can be used to purify a particular peptide, the subject method is based on affinity capture by way of the originally modified amino acid residue after treatment of the peptide with agents that modify other residues in the peptide which might otherwise interfere with the affinity capture of the peptide.

The salient advantage of the subject method is that it can be incorporated in an automated system that reduces the amount of tedious manual labor associated with the traditional method of phosphopeptide analysis. Using methods taught in the prior art, the complete process generally takes at least 2 hours to carry out and requires significant vigilance on the part of the experimentalist. An experienced researcher can generally do no more than 3-4 runs in a day. An automated system (or a series of such systems) can dramatically increase the amount of samples processed per day since most human resource limits are eliminated. Other advantages include:

Efficiency and reproducibility are also increased as the automated components deliver consistent performance not possible with manual methods.

The automated system also allows for multiple column switching abilities. This multiplexing ability can dramatically increase the number of samples analyzed per day.

The incorporation of automated HPLC pumps in the automation process allows the use of gradient elution of the IMAC column, a process not possible by manual methods.

The amount of sample handling is reduced.

The subject method can be illustrated by the example of its use in identifying phosphorylated polypeptides. Phosphopeptides bind Fe(III) with high selectivity, so are amenable to affinity purification using Fe(III) immobilized metal-ion affinity chromatography (IMAC) techniques. However, the presence of hydroxyl and carboxyl groups in the sample peptides, e.g., due to a free carboxyl terminus and the presence of side chains such glutamic acid and aspartic acid, can reduce the efficiency of purification by contributing to non-specific binding to the metal column. Conversion of these side chains to neutral derivatives, such as by alkyl-esterification (which converts Glu and Asp to their neutral, alkyl ester derivatives, and also converts the C-terminal carboxyl group to an alkyl ester) can be used to reduce non-specific binding. The phosphate groups, if any, are not neutralized under the reaction conditions, and are accordingly still available for coordinating a metal ion. Thus, the resulting peptide mixture is contacted with a metal affinity column or resin which retains only peptides which bear the phosphate groups. The other peptides “flow through” the column. The phosphopeptides can then be eluted in a second step and analyzed by mass spectrometry, such as LC/MS/MS. Sequencing of the peptides can reveal both their identity and the site of phosphorylation.

To further illustrate, alkyl esters of free carboxyl groups in a peptide can be formed by reaction with alkyl halides and salts of the carboxylic acids, in an amide-type solvent, particularly dimethylformamide, in the presence of an iodine compound. In other embodiments, the reaction can be carried out with equimolecular amounts of an alkyl halide and a tertiary aliphatic amine.

In yet another embodiment, the method of the present invention can include esterification of the free carboxylic groups by reacting a salt of the carboxylic acid with a halogenated derivative of an aliphatic hydrocarbon, a cycloaliphatic hydrocarbon or an aliphatic hydrocarbon bearing a cyclic substituent in an aqueous medium, and in the presence of a phase transfer catalyst. By the expression “phase transfer catalyst” is intended a catalyst which transfers the carboxylate anion from the aqueous phase into the organic phase. The preferred catalysts for the process of the invention are the onium salts and more particularly quaternary ammonium and/or phosphonium salts.

The alkyl ester of the dipeptide is most preferably a methyl ester and may also be an ethyl ester or alkyl of up to about four carbon atoms such as propyl, isopropyl, butyl or isobutyl.

In still other embodiments, the carboxyl groups can be modified using reagents which are traditionally employed as carboxylprotecting groups or cross-coupling agents, such as 1,3-dicyclohexylcarbodiimide (DCC), 1,1′ carbonyldiimidazole (CDI), 1-ethyl-3-(3-dimethylamiopropyl) carbodiimide hydrochloride (EDC), benzotriazol-1-yl-oxytris(dimethylamino)phosphonium hexafluorophosphate (BOP), and 1,3-Diisopropylcarbodiimide (DICD).

It will be appreciated by those skilled in the art that the subject method can be extended to other types of protein modifications, particularly those which result in modification(s) which change the protein's susceptibility to metal ion affinity purification in a manner dependent on the presence of the modified residues and which difference is enhanced by further chemical modification of other amino acid side chains and/or terminal groups of the protein. Exemplary post-translation modifications for which the subject method can used include glycosylation, acylation, methylation, phosphorylation, sulfation, prenylation, hydroxylation and carboxylation. For example, the automated analysis of glycopeptides could be accomplished by substituting a boronate-type column into the system. Alternatively, a thiol-containing column could be used to purify cysteine-containing peptides. As in the case of phosphorylation, the method can include steps for treating protein samples with agents that selectively react with certain groups that are typically found in peptides (e.g., sulfhydryl, amino, carboxy, hyrdoyl groups and the like).

In certain embodiments, the proteins or protein mixtures are processed, e.g., cleaved either chemically or enzymatically, to reduce the proteins to smaller peptides fragments. In certain preferred embodiments, the amide backbone of the proteins are cleaved through enzymatic digestion, preferably treatment of the proteins with an enzyme which produces a carboxy terminal lysine and/or arginine residue, such as selected from the group of trypsin, Arg-C and Lys-C, or a combination thereof. This digestion step may not be necessary, if the proteins are relatively small.

In certain embodiments, the reactants and reaction conditions can be selected such that differential isotopic labeling can be carried out across multiple different samples to generate substantially chemically identical, but isotopically distinguishable peptides. In this way, the source of particular samples can be encoded in the label. This technique can be used to quantitate differences in phosphorylation patterns and/or levels of phosphorylation between two or more samples. Merely to illustrate, the esterification reaction can be performed on one sample in the matter described above. In another sample, esterification is performed by deuterated or tritiated alkyl alcohols, e.g., D₃COD (D₄ methyl-alcohol), leading to the incorporation of three deuterium atoms instead of hydrogen atoms for each site of esterification. Likewise, ¹⁸O can be incorporated into peptides. The peptide mixtures from the two samples are then mixed and analyzed together, for example by LC/MS/MS. The phoshopeptides will be detected as light and heavy forms, and the relative ratio of peak intensities can be used to calculate the relative ratio of the phosphorylation in the two cases.

It can also be advantageous to perform one methyl-esterification reaction on the whole protein with methyl-alcohol for both samples. Subsequent to enzymatic digestion, one of the samples is then further esterified with D₄ Methyl-alcohol. This leads to the incorporation of three deuterium atoms in each peptide rather than a variable number depending on the number of acidic residues in the peptide.

To complete the analysis, the sample may be further separated by reverse phase chromatography and on-line mass spectrometry analysis using both MS and MS/MS. To illustrate, the sequence of isolated peptides can be determined using tandem MS (MSₙ) techniques, and by application of sequence database searching techniques, the protein from which the sequenced peptide originated can be identified. In general, at least one peptide sequence derived from a protein will be characteristic of that protein and be indicative of its presence in the mixture. Thus, the sequences of the peptides typically provide sufficient information to identify one or more proteins present in a mixture.

In certain other embodiments of the invention, IMAC-bound peptides are eluted by manipulation of the oxidation state of the immobilized metal ion such that the bound peptides have a lower affinity for the resulting oxidation state and, therefore, elute off the column. After elution of the peptides of interest, the IMAC column is regenerated using a suitable redox reagent to return the metal ion to its original oxidation state. For example, the phosphate moiety preferentially binds to iron in a 3⁺ oxidation state (Fe III). Rather than manipulating solution pH in an effort to reduce the binding affinity of phosphate to Fe III, reagents which reduce or oxidize iron to an oxidation state which does not bind phosphate as well can be used. After elution of phosphopeptides, the IMAC column can be regenerated with a suitable redox reagent to return it to a 3⁺ oxidation state.

Such an approach has a number of advantages over current elution methods, which are not ideally suited to subsequent LC-MS and LC-MS/MS analyses. For example, elution of bound phosphopeptides from an IMAC column requires a somewhat basic elution buffer (pH=8-9), and relies on the fact that the phosphate moiety does not compete effectively for activated metal ion binding sites at elevated pH levels. Unfortunately, standard reversed-phase LC packing material (e.g., C₈, C₁₈) does not efficiently capture hydrophilic peptides at basic pH; this is particularly problematic in the case of phosphorylated peptides as the phosphate moiety imparts significant hydrophilic character. As a result careful attention must be paid to buffer pH and elution volume during phosphopeptide analysis by LC-MS and LC-MS/MS. Even then, it is often problematic to analyze various subsets of phosphopeptides.

The use of redox reagents in IMAC chromatography significantly increases the robustness and reproducibility of phosphopeptide analysis. In addition, this approach is more amenable to high throughput phosphopeptide applications. Further, such an elution approach is applicable to any purification protocol which relies upon the interaction of charged species (e.g., ion-exchange chromatography).

To illustrate, ascorbic acid functions in vivo to prevent scurvy by maintaining the iron-center of propyl hydroxylase in its reduced form (Fe²⁺). Thus, once phosphopeptides are bound to an IMAC column, a solution of ascorbic acid may be used to reduce Fe III to Fe II, and thereby facilitate elution of phophopeptides. Moreover, an ascorbic acid elution buffer is somewhat acidic, and thus more amenable to subsequent capture of eluted phophopeptides by standard reversed-phase chromatography. In this configuration, continued elution of phosphopeptides from the IMAC column, coupled in series with a reversed-phase column, may be performed without concern for inefficient elution from the IMAC column or for inefficient capture of phosphopeptides on the reversed-phase column. Again, this methodology may be readily configured for high-throughput applications. After elution of phosphopeptides, the IMAC column may be regenerated (e.g., Fe II→Fe III) by rinsing with a suitable oxidation reagent such as performic acid.

Quantitative relative amounts of proteins in one or more different samples containing protein mixtures (e.g., biological fluids, cell or tissue lysates, etc.) can be determined using isotopic labeling as described above. In this method, each sample to be compared is treated with a different isotopically labeled reagent. The treated samples are then combined, preferably in equal amounts, and the proteins in the combined sample are enzymatically digested, if necessary, to generate peptides. As described above, peptides are isolated by affinity purification based on the post-translation modification of interest and analyzed by MS. The relative amounts of a given protein in each sample is determined by comparing relative abundance of the ions generated from any differentially labeled peptides originating from that protein. More specifically, the method can be applied to screen for and identify proteins which exhibit differential levels of modification in cells, tissue or biological fluids.

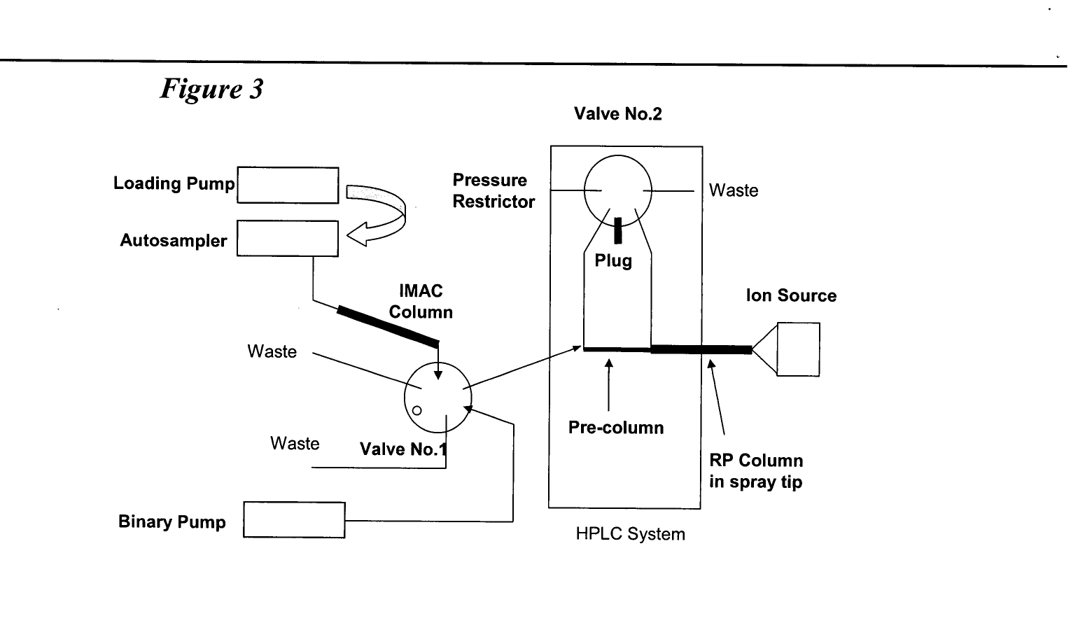

A schematic configuration of equipment which can be used to automate the subject method is shown in FIG. 3. Basic components include an autosampler, a loading pump, two 6-port valves, a binary pump, a pre-column, an IMAC column, and an ion source capable of interfacing with any commercially available mass spectrometer. The autosampler preferably has pre-treatment capability and the ability to hold at least 6 reagent bottles for liquid handling capability. In the illustrate embodiment, the user is only required to prepare the samples and place them in the autosampler.

The method of the present invention is useful for a variety of applications. For example, it permits the identification of enzyme substrates which are modified in response to different environmental cues provided to a cell. Identification of those substrates, in turn, can be used to understand what intracellular signaling pathways are involved in any particular cellular response, as well as to identify the enzyme responsible for catalyzing the modification. To further illustrate, changes in phosphorylation states of substrate proteins can be used to identify kinases and/or phosphatases which are activated or inactivated in a manner dependent on particular cellular cues. In turn, those enzymes can be used as drug screening targets to find agents capable of altering their activity and, therefore, altering the response of the cell to particular environmental cues. So, for example, kinases and/or phosphatases which are activated in transformed (tumor) cells can be identified through their substrates, according to the subject method, and then used to develop anti-proliferative agents which are cytostatic or catatonic to the tumor cell.

In other embodiments, the present method can be used to identify a treatment that can modulate a modification of amino acid in a target protein without any knowledge of the upstream enzymes which produce the modified target protein. By comparing the level of a modification before and after certain treatments, one can identify the specific treatment that leads to a desired change in level of modification to one or more target proteins. To illustrate, one can screen a library of compounds, for example, small chemical compounds from a library, for their ability to induce or inhibit phosphorylation of a target polypeptide. While in other instances, it may be desirable to screen compounds for their ability to induce or inhibit the dephosphorylation of a target polypeptide (i.e., by a phosphatase).

Similar treatments are not limited to small chemical compounds. For example, a large number of known growth factors, cytokines, hormones and any other known agents known to be able to modulate post-translational modifications are also within the scope of the invention.

In addition, treatments are not limited to chemicals. Many other environmental stimuli are also known to be able to cause post-translational modifications. For example, osmotic shock may activate the p38 subfamily of MAPK and induce the phosphorylation of a number of downstream targets. Stress, such as heat shock or cold shock, many activate the JNK/SAPK subfamily of MAPK and induce the phosphorylation of a number of downstream targets. Other treatments such as pH change may also stimulate signaling pathways characterized by post-translational modification of key signaling components.

In another respect, the instant invention also provides a means to characterize the effect of certain treatments, i.e., identifying the specific post-translational modification on specific polypeptides as a result of the treatment.

To illustrate, one may wish to identify the effect of treating cells with a growth factor. More specifically, one may desire to identify the specific signal transduction pathways involved downstream of a growth factor. By comparing post-translational modification levels of certain candidate polypeptides before and after the growth factor treatment, one can use the method of the instant invention to determine precisely what downstream signaling pathways of interest are activated or down regulated. This in turn also leads to the identification of potential drug screen targets if such signaling pathways are to be modulated.

In connection with those methods, the instant invention also provides a method for conducting a drug discovery business, comprising: i) by suitable methods mentioned above, determining the identity of a compound that modulates a modification of amino acid in a target polypeptide; ii) conducting therapeutic profiling of the compound identified in step i), or further analogs thereof, for efficacy and toxicity in animals; and, iii) formulating a pharmaceutical preparation including one or more compounds identified in step ii) as having an acceptable therapeutic profile. Such business method can be further extended by including an additional step of establishing a distribution system for distributing the pharmaceutical preparation for sale, and may optionally include establishing a sales group for marketing the pharmaceutical preparation.

The instant invention also provides a business method comprising: i) by suitable methods mentioned above, determining the identity of a compound that modulates a modification of amino acid in a target polypeptide; ii) licensing, to a third party, the rights for further drug development of compounds that alter the level of modification of the target polypeptide.

The instant invention also provides a business method comprising: i) by suitable methods mentioned above, determining the identity of the polypeptide and the nature of the modification induced by the treatment; ii) licensing, to a third party, the rights for further drug development of compounds that alter the level of modification of the polypeptide.

### EXAMPLE

### Phosphoproteome Analysis by Mass Spectrometry

Sample Preparation. Angiotensin II phosphate was purchased from Sigma and prepared in 0.1% acetic acid solution at a concentration of 100 fmol/μl. A complex biological mixture was obtained by performing a trizol precipitation on a xenograft human glioblastoma. For each sample, aliquots were pressure loaded directly onto an activated IMAC column, and analyzed by mass spectrometry as described below.

Chromatography. Construction of immobilized metal affinity chromatography (IMAC) columns has been described previously (Zarling, et al. Phosphorylated peptides are naturally processed and presented by major histocompatibility complex class I molecules in vivo. J. Exp. Med. 192, 1755-1762 (2000)). Briefly, 360 μm O.D.×100 μm I.D. fused silica (Polymicro Technologies, Phoenix, Ariz.) capillaries, either 360 μm O.D.×100 μm I.D. or 700 μm O.D.×540 μm I.D. were packed with approximately 8 cm POROS 20 MC (PerSeptive Biosystems, Framingham, Mass.). Columns were activated with several hundred microliters of 100 mM FeCl₃ (Aldrich, Milwaukee, Wis.) and pressure loaded with either peptide standards or peptides in complex biological extracts. To remove non-specific binding peptides, the column was washed with a solution containing 100 mM NaCl (Aldrich) in acetonitrile (Mallinkrodt, Paris, Ky.), water, and glacial acetic acid (Aldrich) (25:74:1, v/v/v). For sample analysis by mass spectrometry, the IMAC column was connected to a fused silica pre-column (6 cm of 360 μm O.D.×100 μm I.D.) packed with 5-20 μm C18 particles (YMC, Wilmington, N.C.). All column connections were made with 1 cm of 0.012″ I.D.×0.060″ O.D. Teflon tubing (Zeus, Orangeburg, S.C.). Phosphopeptides were eluted to the pre-column with several hundred microliters of 100 mM ascorbic acid solution (Sigma Chemical Co.); the pre-column was then rinsed with several column volumes of 0.1% acetic acid to remove excess ascorbic acid. The pre-column was connected to the analytical HPLC column (360 μm O.D.×50 or 100 μm I.D. fused silica) packed with 6-8 cm of 5 μm C18 particles (YMC, Wilmington, N.C.). One end of this column contained an integrated laser pulled ESI emitter tip (2-4 μm in diameter)². Sample elution from the HPLC column to the mass spectrometer was accomplished with a gradient consisting of 0.1% acetic acid and acetonitrile.

Mass Spectrometry. All samples were analyzed by nanoflow-HPLC/microelectrospray ionization on a Finnigan LCQ® ion trap (San Jose, Calif.). A gradient consisting of 0-40% B in 60 min, 40-100% B in 5 min (A=100 mM acetic acid in water, B=70% acetonitrile, 100 mM acetic acid in water) flowing at approximately 10 nL/min was used to elute peptides from the reverse-phase column to the mass spectrometer through an integrated electrospray emitter tip (Martin, et al. Subfemtomole MS and MS/MS peptide sequence analysis using nano-HPLC micro-ESI Fourier transform ion cyclotron resonance mass spectrometry. Anal. Chem. 72, 4266-4274 (2000)). Spectra were acquired with the instrument operating in the data-dependent mode throughout the HPLC gradient. Every 12-15 sec, the instrument cycled through acquisition of a full scan mass spectrum and 5 MS/MS spectra (3 Da window; precursor m/z+/−1.5 Da, collision energy set to 40%, dynamic exclusion time of 1 minute) recorded sequentially on the 5 most abundant ions present in the initial MS scan. To perform targeted analysis of the phosphopeptide in the standard mixture, the ion trap mass spectrometer was set to repeat a cycle consisting of a full MS scan followed by an MS/MS scan on the (M+2H)⁺⁺ of DRVpYIHPF (SEQ ID NO: 1) or its ethyl ester analog (m/z 592). The gradient employed for this experiment was 0-100% B in 30 minutes (A=100 mM acetic acid in water, B=70% acetonitrile, 100 mM acetic acid in water).

Database Analysis. All MS/MS spectra recorded on phosphopeptides were searched against a non-redundant protein database using the SEQUEST algorithm. Search parameters included a differential modification of +80 Da (presence or absence of phosphate) on serine, threonine and tyrosine and a static modification of +28 Da (ethyl groups) on aspartic acid, glutamic acid, and the C-terminus of each peptide.

Finally, we note that the above methodology can be modified easily to allow quantitation and/or differential display of phosphoproteins expressed in two different samples. For this experiment, peptides are converted to methyl (or ethyl) esters from one sample with d₀-methanol (or d₀-ethanol) and from the other sample with d₃-methanol (or d₅-ethanol). The two samples are combined, fractionated by IMAC, and the resulting mixture of labeled and unlabeled phosphopeptides is then analyzed by nanoflow HPLC/electrospray ionization. Signals for peptides present in both samples appear as doublets separated by n(3Da)/z (where n=the number of carboxylic acid groups in the peptide and z=the charge on the peptide) or n(5Da)/z. The ratio of the two signals in the doublet changes as a function of expression level of the particular phosphoprotein in each sample. Peptides of interest are then targeted for sequence analysis in a subsequent analysis.

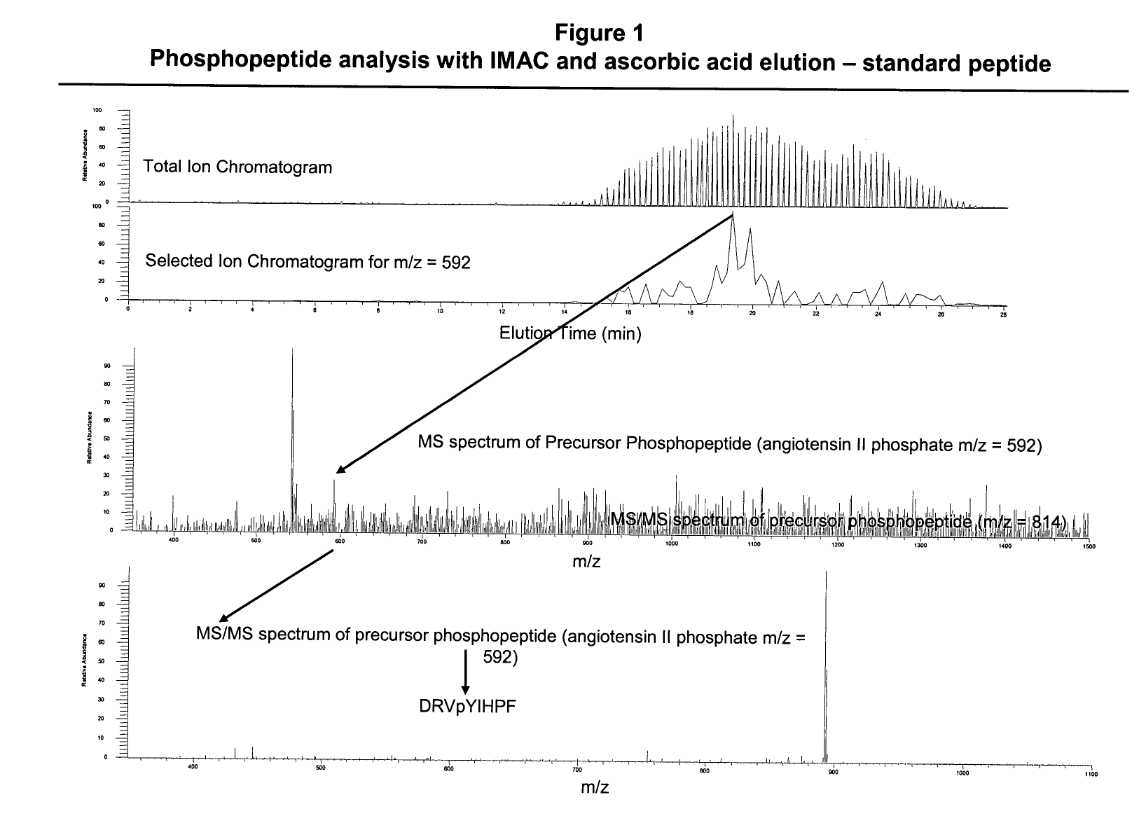

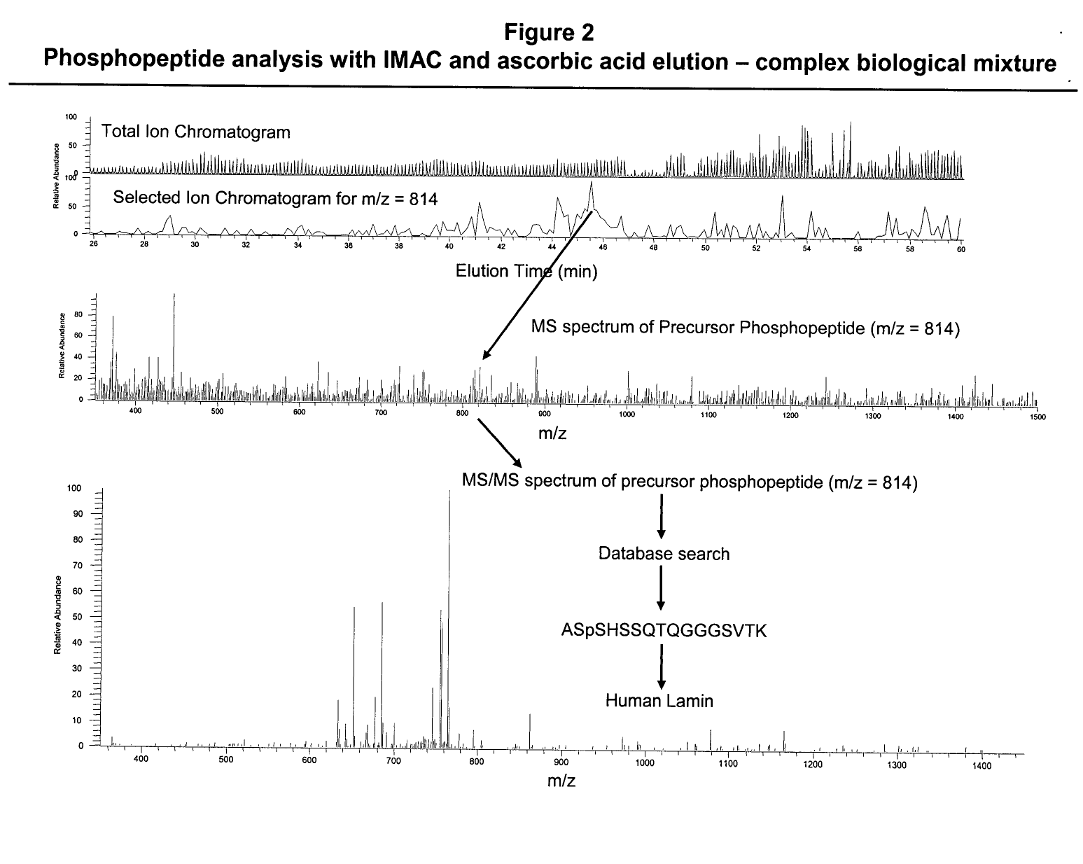

FIGS. 1 and 2 demonstrate the utility of redox chemistry to elute phosphopeptides bound to an IMAC column. In each experiment, peptide mixtures were pressure loaded onto an IMAC column, rinsed, and subsequently eluted from the column directly onto a C₁₈, reversed phase column using 100 mM ascorbic acid solution. Phosphopeptides were gradient eluted from the reversed phase column directly into a quadrupole ion trap mass spectrometer. MS and MS/MS spectra were acquired to verify the presence of phosphopeptides.

FIG. 1 shows data acquired for a simple standard peptide (angiotensin II phosphate).

FIG. 2 shows enrichment of phosphorylated peptides from a complex biological mixture. The data illustrates the MS and MS/MS spectra acquired for a phosphorylated peptide from a human lamin protein.

### REFERENCES

a) Oda, Y., Nagasu, T. & Chait, B. Enrichment analysis of phosphorylated proteins as a tool for probing the phosphoproteome. Nat. Biotechnol. 19, 379-382 (2001).

b) Zhou, H., Watts, J. & Aebersold, R. A systematic approach to the analysis of protein phosphorylation. Nat. Biotechnol. 19, 375-378 (2001).

c) Andersson, L. and Porath, J. Isolation of phosphoproteins by immobilized metal (Fe³⁺) affinity chromatography. Anal. Biochem. 154, 250-254 (1986b).

d) Muszynska, G., Dobrowolska, G., Medin, A., Ekman, P. & Porath, J. O. Model studies on iron(III) ion affinity chromatography. II. Interaction of immobilized nbiron(III) ions with phosphorylated amino acids, peptides and proteins. J. Chrom. 604, 19-28 (1992).

e) Nuwaysir, L. & Stults, J. Electrospray ionization mass spectrometry of phosphopeptides isolated by on-line immobilized metal-ion affinity chromatography. J. Amer. Soc. Mass Spectrom. 4, 662-669 (1993).

We claim:

1. A method for identifying modified amino acids within a protein, comprising:
(i) providing one or more samples and an affinity capture reagent for isolating, from said samples, those proteins post-translationally modified by a moiety of interest;
(ii) processing said samples to chemically modify at least one of the C-terminal carboxyl, the N-terminal amine and amino acid side chains of polypeptides in said samples so as to increase the specificity of said affinity capture reagent for those proteins post-translationally modified by said moiety of interest;
(iii) isolating said proteins post-translationally modified by said moiety of interest from said samples using said affinity capture reagent;
(iv) eluting said proteins bound to said affinity capture reagent by manipulating the oxidation state of said affinity capture reagent; and,
(v) determining the identity of said proteins eluted in (iv) by mass spectroscopy.

2. The method of claim 1, wherein said polypeptides in said samples are further cleaved into smaller peptide fragments before, after or during the step of processing said samples.

3. The method of claim 2, wherein said polypeptides are fragmented by enzymatic hydrolysis to produce peptide fragments having carboxy-terminal lysine or arginine residues.

4. The method of claim 3, wherein said polypeptides are fragmented by treatment with trypsin.

5. The method of claim 1, wherein said polypeptides are mass-modified with isotopic labels before, after or during the step of processing said samples.

6. The method of claim 1, wherein said proteins isolated in steps (iii)/(iv) are further separated by reverse phase chromatography before analysis by mass spectroscopy.

7. The method of claim 1, wherein said proteins isolated in steps (iii) and (iv) are identified from analysis using tandem mass spectroscopy techniques.

8. The method of claim 1, wherein step (v) is effectuated by searching molecular weight databases for the molecular weight observed by mass spectroscopy for an isolated protein or peptide fragment thereof.

9. The method of claim 1 or 7, further comprising obtaining amino acid sequence mass spectra for said proteins or peptide fragments thereof, and searching one or more sequence databases for the sequence(s) observed for said protein or peptide fragments thereof.

10. The method of claim 1, wherein said moiety of interest is a phosphate group.

11. The method of claim 10, wherein said affinity capture reagent is an immobilized metal affinity chromatography medium, and step (ii) includes chemically modifying the side chains of glutamic acid and aspartic acid residues to neutral derivatives.

12. The method of claim 11, wherein the side chains of glutamic acid and aspartic acid residues are modified by alkyl-esterification.

13. The method of claim 1, wherein said sample comprises a mixture of different proteins.

14. The method of claim 13, wherein said sample is derived from a biological fluid, or a cell or tissue lysate.

15. The method of claim 1, wherein said one or more samples comprise two or more different samples, and wherein the polypeptides or fragments thereof of each sample are isotopically labeled in a manner which permits discrimination of mass spectroscopy data between different samples.

16. A method for analyzing a phosphoproteome, comprising:
(i) providing one or more protein sample(s);
(ii) chemically modifying the side chains of glutamic acid and aspartic acid residues of polypeptides in said protein sample(s) to neutral derivatives;
(ii) isolating phosphorylated proteins from said protein sample(s) by using immobilized metal affinity chromatography;
(iii) eluting said phosphorylated proteins from said affinity capture reagent by manipulating the oxidation state of said reagent; and,
(iv) determining the identity of said phosphorylated p roteins eluted in (iii) by m ass spectroscopy.

17. The method of claim 16, further comprising cleaving said polypeptides into smaller peptide fragments, before, after or during the step of chemically modifying the glutamic acid and aspartic acid residues.

18. The method of claim 17, wherein said polypeptides are fragmented by enzymatic hydrolysis to produce peptide fragments having carboxy-terminal lysine or arginine residues.

19. The method of claim 18, wherein said polypeptides are fragmented by treatment with trypsin.

20. The method of claim 16, wherein the glutamic acid and aspartic acid residues are modified by alkyl-esterification.

21. The method of claim 16, wherein said one or more sample(s) comprise two or more different samples, the method further comprises identifying proteins which are differentially phosphorylated between said two or more different samples.

22. The method of claim 16 or 21, further comprising generating or adding to a database the identity of proteins which are determined to be phosphorylated.

23. A method for identifying a treatment that modulates a modification of amino acid in a target polypeptide, comprising:
(i) providing a sample which has been subjected to a treatment of interest;
(ii) determining, using the method of claim 1, the identity of proteins which are differentially modified in said treated sample relative to an untreated sample or control sample;
(iii) determining, whether said treatment results in a pattern of changes in protein modification which meets a preselected criterion, in said treated sample relative to said untreated sample or control sample.

24. The method of claim 23, wherein said treatment is effected by a compound.

25. The method of claim 24, wherein said compound is a growth factor, a cytokine, a hormone, or a small chemical molecule.

26. The method of claim 24, wherein said compound is from a chemical library.

27. The method of claim 23, wherein said sample is derived from a cell or tissue subjected to said treatment of interest.

28. A method of conducting a drug discovery business, comprising:
(i) determining, by the method of claim 24, the identity of a compound that produces a pattern of changes in protein modification which meet a preselected criterion, in said treated sample relative to said untreated sample or control sample;
(ii) conducting therapeutic profiling of said compound identified in step (i), or further analogs thereof, for efficacy and toxicity in animals; and,
(iii) formulating a pharmaceutical preparation including one or more compound(s) identified in step (ii) as having an acceptable therapeutic profile.

29. The method of claim 28, including an additional step of establishing a distribution system for distributing the pharmaceutical preparation for sale, and may optionally include establishing a sales group for marketing the pharmaceutical preparation.

30. A method of conducting a drug discovery business, comprising:
(i) determining, by the method of claim 24, the identity of a compound that produces a pattern of changes in protein modification which meet a preselected criterion, in said treated sample relative to said untreated sample or control sample;
(ii) licensing, to a third party, the rights for further drug development of compounds that alter the level of modification of the target polypeptide.

31. A method of conducting a drug discovery business, comprising:
(i) by the method of claim 1, determining the identity of a protein that is post-translationally modified under conditions of interest;
(ii) identify one or more enzymes which catalyze the post-translational modification of the identified protein under the conditions of interest;
(iii) conduct drug screening assays to identify compounds which inhibit or potentiate the enzymes identified in step (ii) and which modulate the post-translational modification of the identified protein under the conditions of interest.


SEQUENCE LISTING (2 sequences)

SEQ ID NO: 1 - protein, 8 aa, Homo sapien
  PHOSPHORYLATION 4: Angiotensin II
DRVYIHPF

SEQ ID NO: 2 - protein, 16 aa, Homo sapien
  PHOSPHORYLATION 3: Angiotensin II
ASSHSSQTQGGGSVTK